(19) United States
(12) Patent Application Publication
(10) Pub. No.: US 2011/0082452 A1
(43) Pub. Date: Apr. 7, 2011

(54) CARDIAC ABLATION SYSTEM WITH AUTOMATIC SAFETY SHUT-OFF FEATURE

(75) Inventors: Gerald Melsky, Lexington, MA (US); Stephen W. Sagon, Amherst, NH (US)

(73) Assignee: CardioFocus, Inc., Marlborough, MA (US)

(21) Appl. No.: 12/896,010

(22) Filed: Oct. 1, 2010

Related U.S. Application Data
(60) Provisional application No. 61/248,195, filed on Oct. 2, 2009.

(51) Int. Cl. A61B 18/24

(52) U.S. Cl. 606/15

(57) ABSTRACT

A cardiac tissue ablation system according to one embodiment includes an elongate catheter configured to deliver a distal end thereof to a patient's heart and an expandable member formed at the distal portion of the catheter. The expandable member has an elastic portion configured to conform to the shape of a target tissue region upon expansion. The system further includes an energy emitter movably disposed within a lumen of the catheter. The energy emitter is configured to deliver radiant ablative energy to the target tissue region. The system further includes a controller that includes a safety shut-off feature for automatically shutting off power to the energy emitter when a prescribed event occurs resulting in power to the energy emitter being stopped.

### CROSS REFERENCE TO RELATED APPLICATIONS

The present application claims the benefit of U.S. patent application Ser. No. 61/248,195, filed Oct. 2, 2009 which is hereby incorporated by reference in its entirety.

### TECHNICAL FIELD

The present disclosure relates to ablation instruments and methods of use thereof, in particular to automatic safety shut-off features that are actuated upon the occurrence of a prescribed event.

### BACKGROUND

Cardiac arrhythmias, e.g., fibrillation, are irregularities in the normal beating pattern of the heart and can manifest themselves in either the atria or the ventricles of the heart. For example, atrial fibrillation is a form of arrhythmia characterized by rapid randomized contractions of atrial myocardium, causing an irregular, often rapid ventricular rate. The regular pumping function of the atria is replaced by a disorganized, ineffective quivering as a result of chaotic conduction of electrical signals through the upper chambers of the heart. Atrial fibrillation is often associated with other forms of cardiovascular disease, including congestive heart failure, rheumatic heart disease, coronary artery disease, left ventricular hypertrophy, cardiomyopathy, or hypertension.

Effective catheter treatment of atrial fibrillation was made possible by a breakthrough discovery in the late 1990's by investigators in Bordeaux, France. They found that recurrent atrial fibrillation (paroxysmal and persistent) is triggered by rapidly firing tissue, (called “ectopic foci”), that are located in one or more of the four pulmonary veins, which attach to the rear of the left atrium. Their research, since confirmed by others, suggested that 85% or more of the ectopic foci that initiate atrial fibrillation are located in or at the ostium, (mouth), of the pulmonary veins. They demonstrated that atrial fibrillation could be cured by electrically isolating the pulmonary veins from the rest of the atrium.

Various techniques have been proposed for pulmonary vein isolation. Although these procedures were originally performed with a scalpel, various other techniques have also been developed to form lesions. Collectively, these treatments are referred to as “ablation.” In non-surgical ablations, the tissue is treated, generally with heat or cold, to cause coagulation and/or tissue necrosis (i.e., cell destruction). In each of these techniques, cardiac muscle cells are replaced with scar tissue which cannot conduct normal electrical activity within the heart.

In one known approach, circumferential ablation of tissue within the pulmonary veins or at the ostia of such veins has been practiced to treat atrial fibrillation. By ablating the heart tissue at selected locations, electrical conductivity from one segment to another can be blocked and the resulting segments become too small to sustain the fibrillatory process on their own.

Several types of ablation devices have recently been proposed for creating lesions to treat cardiac arrhythmias, including devices which employ electrical current (e.g., radio-frequency (“RF”)) heating or cryogenic cooling. Such ablation devices have been proposed to create elongated lesions that extend through a sufficient thickness of the myocardium to block electrical conduction. Many of the recently proposed ablation instruments are percutaneous devices that are designed to create such lesions from within the heart. Such devices are positioned in the heart by catheterization of the patient, e.g., by passing the ablation instrument into the heart via a blood vessel, such as the femoral vein.

Devices that rely upon resistive or conductive heat transfer can be prone to serious post-operative complications. In order to quickly perform an ablation with such “contact” devices, a significant amount of energy must be applied directly to the target tissue site. In order to achieve transmural penetration, the surface that is contacted will experience a greater degree of heating (or freezing). For example, in RF heating of the heart wall, a transmural lesion requires that the tissue temperature be raised to about 50° C. throughout the thickness of the wall. To achieve this, the temperature at the contact surface will typically be raised to greater than 100° C. In this temperature regime, there is a substantial risk of tissue destruction (e.g., due to water vaporization micro-explosions or due to carbonization). Charring of the surface of the heart tissue, in particular, can lead to the creation of blood clots on the surface and post-operative complications, including stroke. Even if structural damage is avoided, the extent of the lesion (i.e., the width of the ablated zone) on the surface that has been contacted will typically be greater than necessary.

Cardiac ablation instruments also suffer from a variety of design limitations. For example, the shape of the heart muscle adds to the difficulty in accessing cardiac structures, such as the pulmonary veins on the anterior surface of the heart. Typically, percutaneous devices are positioned with the assistance of a guide wire, which is first advanced into heart. In one common approach, described, for example, in U.S. Pat. No. 6,012,457 issued to Lesh on Jan. 11, 2000 and in International Application Pub. No. WO 00/67656 assigned to Atrionix, Inc., a guide wire or similar guide device is advanced through the left atrium of the heart and into a pulmonary vein. A catheter instrument with an expandable element is then advanced over the guide wire and into the pulmonary vein where the expandable element (e.g., a balloon) is inflated. The balloon includes a circumferential ablation element, e.g., an RF electrode, carried on the outer surface of the balloon, which performs the ablation procedure. The balloon must be large enough and sufficiently rigid to hold the electrode in contact with the inner surface of the pulmonary vein for the length of the procedure. Moreover, because the lesion is formed by an ablation element carried on the surface of the balloon element, the balloon shape inherently limits the locations where a lesion can be formed, i.e., the lesion must be formed at least partially within the pulmonary vein.

In another approach described in U.S. Pat. No. 6,235,025 issued to Swartz et al. on May 22, 2001, a guide wire is again used to percutaneously access a pulmonary vein and a catheter is again slid over the guide wire to a position within the pulmonary vein. The catheter device includes two spaced-apart balloons, which are inflated in the vein (or in the vein and at its mouth). The space between the two balloons can then be filled with a conductive fluid to delivery RF energy (or, alternatively, ultrasound) to the vein and thereby induce a conduction block in the blood vessel by tissue ablation. With the Swartz et al. device, like the Lesh device, the region where tissue ablation can occur is limited by the design. Because two balloons must seal a space that is then filled with an ablative fluid, the lesion is necessarily formed within the pulmonary vein.

Ablation within the pulmonary vein can result in complications. Overtreatment deep within a vein can result in stenosis (closure of the vein itself), necrosis or other structural damage, any of which can necessitate immediate open chest surgery.

A limitation of these commonly utilized instruments is the lack of site selectability. Practically speaking, each such percutaneous instrument is inherently limited by its design to forming an ablative lesion at one and only one location. For example, when an expandable balloon carrying an RF heating device on its surface is deployed at the mouth of a vein, the lesion can only be formed at a location defined by the geometry of the device. It is not possible to form the lesion at another location because the heating element must contact the target tissue. Similarly, the above-described tandem balloon device can only form a lesion at a location defined by the space between the balloons that is filled with the ablative fluid.

Another limitation of such known instruments and methods is their inability to accommodate the varied geometry of the heart. For example, the inner surface of the atrium is not regular. In particular, the mouths of the.pulmonary veins do not exhibit regularity; they often bear little resemblance to conical or funnel-shaped openings. Thus, when the above-described expandable, contact heating devices encounter an irregularly-shaped ostia, the result can be an incompletely formed (non-circumferential) lesion.

Moreover, the size or shape of the pulmonary vein ostia that are encountered may be too big or too small for the selected ballooti catheter and it may be necessary to remove the first balloon catheter from the patient and replace it with another instrument having a balloon element of a different size. Replacement of the catheter with another before a procedure can begin (or in the middle of a multiple vein ablation protocol) can substantially increase the overall duration of the procedure and/or increase the chance of trauma.

Accordingly, there exists a need for better cardiac ablation instruments that can quickly and effective create pulmonary vein encircling lesions even in the face of irregularly shaped or variable sized target tissue regions.

### SUMMARY

Methods and instruments are disclosed for creating lesions in tissue, especially cardiac tissue, for treatment of arrhythmias and the like, by employing an elastic balloon and an energy emitter, which is independently positionable within the lumen of the instrument and adapted to project a series of spots of ablative energy through a transmissive region of the balloon to a target tissue site. The energy emitter preferably is configured such that the spots of energy result in a series of lesions formed in the target tissue region when the emitter is activated, the lesions having an average area ranging from about 5 mm² toabout 100 mm² and, in some instances, 10 mm² to about 80 mm² In certain embodiments, the energy emitter is also be configured to form arc shaped lesions in the target tissue region, each arc shaped lesion subtending an angle ranging from about 5 degrees to about 120 degrees, preferably less than 30 degrees relative to the rotatable emitter (e.g., based on the emitter or longitudinal axis of optical element serving as the center of a circular frame of reference).

In one aspect of the invention, percutaneous ablation instruments are disclosed in the form of catheter bodies having one or more balloon structures at the distal end region of the instrument. The balloon structure and catheter bodies are at least partially (and preferably completely) transmissive to ablative energy. The instruments can further include an energy emitting element, which is independently positionable and rotatable within the lumen of the instrument and adapted to project ablative energy through a transmissive region of the balloon to a target tissue site in contact with the balloon surface. The energy is delivered without the need for contact between the energy emitter and the target tissue because the methods and devices of the present invention do not rely upon conductive or resistive heating. Because the position of the radiant energy emitter can be varied, the clinician can select the location of the desired lesion.

The present invention provides a mechanism for addressing the problem of instrument orientation and/or irregularly-shaped pulmonary veins by rotation and, in some instances, adjustment of the location of the energy emitter to form a series of spot lesions that overlap and create a circumferential block. For example, the devices of the present invention can form a first series of lesions along a first arc when the energy emitter is in a first location and a second series of lesions along a second arc when the energy emitter is in a second location. The spot lesions can be combined to form a continuous encircling or circumscribing lesion that blocks fibrillation-inducing electric conduction.

Monitoring allows the clinician to observe balloon inflation and establish an optimal size (with sufficient contact for ablation). Reflective monitoring and/or imaging, especially with wide field of view optics, can be used to determine if the instrument is too deep within a pulmonary vein by mapping tissue contact on the balloon. For example, tissue contact with proximal (rear) portions of the balloon can indicate over-insertion of the balloon in to a vein. Because the device has a wide angle field of view and a large depth of field the clinician can also see the relationship of the balloon to the pulmonary vein ostia. This capability allows the clinician to determine if the balloon is too deep in the pulmonary vein because the optical sensor has the ability to see a large portion of the interior the balloon. For example, if a large extent of tissue contact is observed (or otherwise sensed) proximal to the equator of the balloon (the circumferential zone of greatest diameter), this can indicate that the instrument is too deep in the vein and the creation of lesions in this configuration may pose a risk of causing pulmonary vein stenosis. One exemplary type of visualization instrument is an endoscope.

In addition to determining the degree of contact between the instrument and the tissue, the endoscope can be used to determine the extent of tissue ablation by sensing the change in color of the tissue as it is ablated. Moreover, the endoscope can be used to detect the formation of potentially dangerous coagulated blood at the site of ablation and to allow the clinician to terminate the ablation if necessary for safety reasons. The endoscopic image can also be used to extract colorimetric data, e.g., to distinguish between tissue and blood.

In accordance with one embodiment, a method for treating a cardiac condition with visual inspection of a tissue treatment site includes the step of delivering an expanding member formed on a distal end of a catheter to a position adjacent a tissue treatment area within a patient's heart. The expandable member has an elastic portion configured to conform to the tissue treatment area. The method further includes the steps of expanding the expandable member thereby allowing the elastic portion of the expandable member to conform to the tissue treatment area and positioning an energy emitter at a first location within an inner lumen of the catheter. A visible aiming beam is projected during positioning of the energy emitter and ablative energy is delivered from the energy emitter to the tissue treatment area thereby resulting in a first spot lesion. The ablative energy is coincident with the aiming beam and the aiming beam is in the form of pulsed visible light to allow intermittent visual inspection of the first spot lesion at times when the aiming beam is not projected onto the tissue treatment area, thereby allowing visual inspection of the ablative characteristics and sufficiency of the first spot lesion.

In accordance with the present invention, the balloon is of a multi-stage inflation type in that the balloon can be inflated to a plurality of different inflation settings depending upon the particular application and the landscape of the target site. The landscapes of the hearts of different patients can vary greatly and therefore, it is very difficult to use a single size, single shaped balloon to perform a procedure for all patients. In addition, there is a degree of variability within a single patient that prevents a single instrument from typically being used and in particular, the constructions (shape and sizes) of the different veins of the patient vary and prevent a single instrument from being used. More specifically, the contours (landscape) of the atrial heart wall surrounding the ostium of a pulmonary vein vary greatly from person to person such that it can be very difficult to position an inflated balloon in this area to perform a procedure. Inflatable balloons that are used in conventional cardiac ablation instruments are of a single size such that the balloon is inflated and then positioned along the atrail heart wall. Unfortunately, due to the uneven and irregular contours of this target ablation region, proper and desired contact (seating) between the balloon and the target tissue is difficult. The conventional devices are very limiting since they do not account for different patient heart contours and thus, multiple sized devices are sold.

In contrast, the inflatable balloon of the present invention is configured as a multi-stage balloon in that the inflation thereof can be precisely controlled and the user can effectively “dial” or select an inflation setting that best serves the particular patient based on information contemporaneously obtained with the endoscope regarding the landscape of the atrial heart wall against which the balloon is to be positioned. In other words, there are multiple inflation settings for the balloon to accommodate the wide range of different contours of the atrial walls of different patients. In patients were access is difficult, the balloon can be inflated to a lesser setting to allow the balloon to occupy less area and be more compliant to allow for position against the target tissue. Conversely, in other settings, the balloon can be inflated to a greater degree resulting in greater inflated balloon that occupies a greater space and has a different degree of conformity to permit the proper seating of the balloon against the landscape of the atrial heart wall.

The operation of the multi-stage inflatable balloon can be by any number of different controllers that permit the user to select a particular setting after the landscape of the atrial wall is investigated as described below. For example, the controller can be in the form of a switch that has multiple positions that correspond to multiple inflation settings and therefore, to select a particular setting, the user simply manipulates the switch and places it in the appropriate desired position. Alternatively, the controller can be in the form of a knob that can be rotated into a plurality of different positions which correspond with multiple inflation settings. In addition, the controller can be part of a digital display unit where the user can use a keypad or the like to enter a particular setting that corresponds with one inflation setting. For example, the settings can be identified by different inflation setting numbers or can be identified by different pressure settings. It will be understood that the user can easily change the selected setting by readjustment of the controller to cause the balloon to be inflated at a different inflation setting (level).

By having a multi-stage inflation balloon, the balloon of the present invention can be part of a “one size fits all” balloon system, and, the inflation of the balloon can be customized or tailored to a particular user. This permits a single balloon to be able to be used for all patients and greatly simplifies the process and further, yields to much improved results due to better contact between the balloon and the target area.

The present invention provides mechanisms for visualizing where the energy will be applied prior to treatment. In one embodiment, the energy delivering element can include an aiming light source to project visible light onto the tissue, and reflected light can be observed via the endoscope. This visible light from the aiming beam provides an indication to the clinician of the exact location of energy delivery. Markers on the balloon are also disclosed for such visualization based on known correlations between the variable position of the energy source and particular regions of the balloon. In another embodiment, virtual markers are employed. Based on such visual data, the location of ablation element can be selected to optimize the lesion formed upon activation of the ablation element.

Since the energy delivered by the energy emitter is typically not visible, the aiming light which is coincident therewith assists the user as described above; however, when the aiming light continuously illuminates the tissue, it also obscures the view of the tissue. In particular, the aiming light illuminates the tissue and makes it difficult to notice a change in appearance of the treated tissue that is indicative of the tissue being properly ablated. It is also thus difficult to see the progress of the ablation treatment as the physician treats different tissue areas. In accordance with the present invention, the aiming light has a pulsed operating mode in which the visible light from the aiming light unit is delivered in pulses to cause intermittent illumination of the tissue. In other words, the aiming light has the appearance of being a blinking light. In one embodiment, the aiming light has a green appearance; however, this is merely one exemplary application and it will be understood that the aiming light can have any number of different colors that appear in the visible spectrum. By delivering the visible aiming light in pulses, the operator is able to directly observe the tissue that is being treated at times when the beam is off. In between the blinking of the aiming light, the operator can observe the treated tissue and see how the treatment is progressing by means of an endoscope or the like since the treatment site and field of view of the endoscope is illuminated by other means, including illumination fibers as discussed herein. By having colored pulses of visible light, the visible light of the aiming light can be distinguished from the other light used in the system, such as light from illumination fibers and the like. The lesion quality can thus be readily observed with the visualization system (endoscope).

If the operator is not satisfied with the nature of the ablation being formed, the operator can simply redo or continue the treatment of a specific tissue location. Conversely, if the operator is satisfied with the treatment, the operator can move on to a new tissue location to continue the treatment. It will be appreciated that the ablative energy from the energy emitter can be operated in a continuous mode where the ablative energy is continuously delivered to the tissue or can be operated in a pulsed mode, similar to the aiming light, where ablative energy is delivered in pulses. The pulses of ablative energy can be coincident with the pulses of visible light or the pulses can be set off from one another.

A cardiac tissue ablation system according to one embodiment includes an elongate catheter configured to deliver a distal end thereof to a patient's heart and an expandable member formed at the distal portion of the catheter. The expandable member has an elastic portion configured to conform to the shape of a target tissue region upon expansion. The system further includes an energy emitter movably disposed within a lumen of the catheter. The energy emitter is configured to deliver radiant ablative energy to the target tissue region. The system further includes a controller that includes a safety shut-off feature for automatically shutting off power to the energy emitter when a prescribed event occurs resulting in power to the energy emitter being stopped.

### BRIEF DESCRIPTION OF THE DRAWINGS

The invention will be more fully understood from the following detailed description taken in conjunction with the accompanying drawings, in which like reference numerals designate like parts throughout the figures, and wherein:

FIG. 1 is a schematic, partially-cutaway view of a cardiac ablation instrument according to the invention;

FIG. 2A is a schematic view of the cardiac ablation instrument of FIG. 1 in a treatment position at the ostium of pulmonary vein;

FIG. 2B is a schematic view of the cardiac ablation instrument of FIG. 1 with its compliant balloon inflated and its ablation element deployed at one of a plurality of locations;

FIG. 3 is a schematic illustration of an initial step in performing ablative surgery according to the invention, in which a guide wire is percutaneously inserted into the heart;

FIG. 4 is a schematic illustration of a further step in performing ablative surgery in which a deflectable sheath catheter is deployed in the heart over the guide wire;

FIG. 5 is a schematic illustration of a further step in performing ablative surgery in which a deflectable sheath catheter is maneuvered into a position proximal to a pulmonary vein and the guide wire is withdrawn and replaced with an ablation catheter according to the invention;

FIG. 6 is a schematic illustration of a further step in performing ablative surgery in which a balloon structure of the present invention is inflated at the ostium of a pulmonary vein;

FIG. 7 is a schematic view of the cardiac ablation instrument with its compliant balloon inflated and illustrating how its ablation element can be deployed at different locations to direct radiant energy through different regions of the balloon when an asymmetric vein mouth is encountered and further showing how the position of the radiant energy emitter can be adjusted to select a desired location;

FIG. 8 illustrates how a continuous, vein-encircling lesion can be formed by a series of overlapping spot lesions;

FIG. 9 is a schematic block diagram of the components of an endoscope-guided cardiac ablation system according to the invention;

FIG. 10 is a schematic illustration of one embodiment of a radiant light energy emitter according to the invention;

FIG. 11 is a schematic exploded view of the optical elements of the emitter of FIG. 10;

FIG. 12 is a schematic illustration of another embodiment of a radiant light energy emitter according to the invention in which the elements are configured to project a arc-shaped spot of ablative energy;

FIG. 13 is a schematic illustration of another embodiment of a radiant light energy emitter according to the invention;

FIGS. 14A and 14B show arc-shaped spots of ablative energy;

FIG. 15 is a view of an ultrasound actuator and optical analyzer;

FIG. 16 is a schematic cross-sectional view of an endoscope useful in the present invention;

FIG. 17 is a screen shot of a catheter setup screen showing controls for the balloon and other controls; and

FIG. 18 is a pull down menu for selecting the stage or level of balloon inflation.

### DETAILED DESCRIPTION

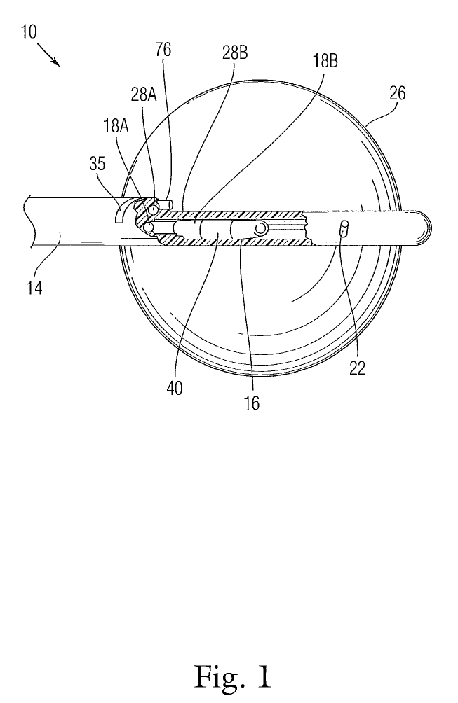

FIG. 1 provides a schematic, cross-sectional view of an ablation instrument 10 according to the invention, including an elongate body 14, a central lumen tubing 16 and a compliant balloon 26 inflatable via one or more ports 22 in the central tubing. The central tubing 16 can also house an energy emitter that is capable of both axial movement and rotation within the tubing 12. Within the elongated body (also referred to herein as the catheter body) there can be a plurality of additional lumens, through which certain devices or instruments can been passed. For example, as shown in FIG. 1, the catheter body 14 also provides lumens 18A and 18B for extraction (or circulation) of a inflation fluid, an endoscope 76 and illuminations fibers 28A and 28B. The catheter body can carry a marker to assist the clinician in proper placement of the device, e.g., a radiopaque marker to aid in fluoroscopic detection.

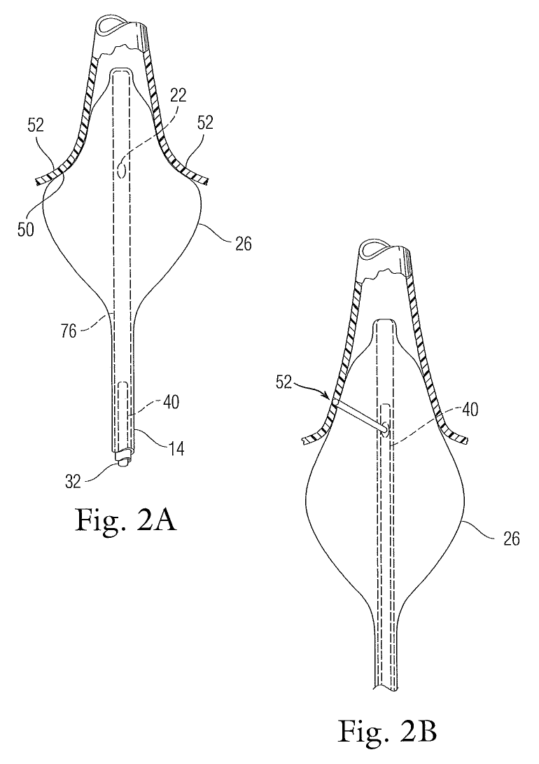

As shown in FIG. 2A, the instrument is preferably designed such that upon disposition within the heart (e.g., proximal to a pulmonary vein), the balloon can be inflated such that a shoulder portion 50 of the balloon 26 will be urged into close proximity with a target region 52 of cardiac tissue (e.g. an region of the atrial heart wall surrounding the ostium of a pulmonary vein). As shown in FIG. 2B. the energy emitter (or “lesion generator') 40 can be positioned to delivery ablative energy 21 to the target region 52 to form a lesion.

It should be understood that the embodiments illustrated in the drawings are only a few of the cardiac ablation instruments that can utilized the present invention. Further descriptions of other embodiments can be found, for example, in commonly owned, co-pending U.S. patent application Ser. No. 10/357,156, filed Feb. 3, 2003 and U.S. patent application Ser. No. 09/924,393, filed Aug. 7, 2001, both of which are expressly incorporated by reference.

The instrument can optionally include one or more ports for delivering irrigation fluid to the target region. When the device employs radiant energy ablation, the fluid is preferably an energy transmissive medium, which helps deliver light, radiation or acoustic energy from a radiant energy source to a target tissue region. The fluid also serves to clear blood from the vicinity of the instrument and compensate for irregularities in the shape of the heart that might otherwise compromise the seating of the instrument. The ablative fluid thus provides a clear transmission pathway external to the balloon.

Returning to FIG. 2B, a radiant energy emitter 40 is shown disposed within the projection balloon 26 remotely from the target tissue (e.g., within a central lumen of the catheter body 14 or otherwise disposed within the balloon). In one embodiment, the radiant energy source includes at least one optical fiber coupled to a distal light projecting, optical element, which cooperate to project a spot of ablative light energy through the instrument to the target site. The catheter body, projection balloon and inflation/ablation fluids are all preferably substantially transparent to the radiant energy at the selected wavelength to provide a low-loss transmission pathway from the ablation element 40 to the target.

Also disposed within the instrument is a reflectance sensor, preferably an endoscope 76 capable of capturing an image of the target site and/or the instrument position. The endoscope is typically an optical fiber bundle with a lens or other optical coupler at its distal end to receive light. The reflectance sensor/endoscope can also include an illumination source, such one or more optical fibers coupled to a light source or sources. Endoscopes are available commercially from various sources. The endoscope can further include an optical head assembly, as detailed in more detail below, to increase the field of view. In one preferable embodiment, ablation element 40 and endoscope 76 are adapted for independent axial movement within the catheter body 14.

As described herein, the endoscope 76 can be used to present the user with feedback as to the quality of the contact between the distal end of the balloon and the target area (atrial heart wall). A rating system or the like can be provided for evaluating the quality of contact between the balloon and the target area. For example, a point system, such as a rating between 1-5, can be provided to give the user a value that allows the user to judge the quality of the contact between the balloon and the target area. In effect, information from the endoscope 76 can be used to tell if the balloon has successfully landed on the target area.

In addition, the information from the endoscope 76 allows evaluation of candidate balloon-contact locations. This investigation will allow the user to determine the degree of inflation of the balloon based on information obtained from the endoscope 76. In other words, by illuminating and then viewing the target area by means of the endoscope 76, the user can view the landscape of this particular patient and select an appropriate inflation level that best complements the observed landscape.

The term “endoscope” as used herein is intended to encompass optical imaging devices, generally, including but not limited to endoscopes, fiberscopes, cardioscopes, angioscopes and other optical fiber-based imaging devices. More generally, “endoscope” encompasses any light-guiding (or waveguide) structure capable of transmitting an “image” of a object to a location for viewing. The viewing location can be direct, e.g., an eyepiece, or indirect, e.g., an image capture device, such as a CCD camera, which converts image data into a video display.

The endoscope 76 is purposely placed relative to the illumination fibers 28A, 28B so as to optimize the field of view of the endoscope 76 and optimize the imaging environment. In particular, ends of the illumination fibers 28A, 28B are placed behind the endoscope 76 so that light emanating from the illumination fibers 28A, 28B does not interfere with the field of view of the endoscope 76. In other words, the field of view of the endoscope 76 does not include the ends of the'illumination fibers 28A, 28B and therefore, the real-time image obtained from the endoscope 76 does not include a pair of “bright spots” that represent the two illumination ends of the illumination fibers 28A, 28B. The placement of the illumination fibers 28A, 28B provides proper backlighting of the endoscope 76 and permits optimal lighting conditions for use with the endoscope 76 to thereby allow optimal lighting of the tissue to be treated.

In certain preferred embodiments, the balloon is a compliant balloon, e.g. an elastic balloon which is expandable to a variable volume to accommodate pulmonary veins (or other target sites of various sizes, thereby alleviating the need to deploy a balloon catheter instrument based on an estimated or assumed vein size and then remove and redeploy another instrument if the cardiac anatomy is different than predicted at the outset.

In accordance with the present invention, the balloon is of a multi-stage inflation type in that the balloon can be inflated to a plurality of different inflation settings depending upon the particular application and the landscape of the target site. The landscapes of the hearts of different patients can vary greatly and therefore, it is very difficult to use a single size, single shaped balloon to perform a procedure for all patients. More specifically, the contours (landscape) of the atrail heart wall surrounding the ostium of a pulmonary vein vary greatly from person to person such that it can be very difficult to position an inflated balloon in this area to perform a procedure. Inflatable balloons that are used in conventional cardiac ablation instruments are of a single size such that the balloon is inflated and then positioned along the atrail heart wall. Unfortunately, due to the uneven and irregular contours of this target ablation region, proper and desired contact (seating) between the balloon and the target tissue is difficult. The conventional devices are very limiting since they do not account for different patient heart contours and thus, they are sold with a “one size fits all” mentality.

In contrast, the inflatable balloon of the present invention is configured as a multi-stage balloon in that the inflation thereof can be precisely controlled and the user can effectively “dial” or select an inflation setting that best serves the particular patient based on information previously obtained regarding the landscape of the atrial heart wall against which the balloon is to be positioned. In other words, there are multiple inflation settings for the balloon to accommodate the wide range of different contours of the atrial walls of different patients. In patients were access is difficult, the balloon can be inflated to a lesser setting to allow the balloon to occupy less area and be more compliant to allow for position against the target tissue. Conversely, in other settings, the balloon can be inflated to a greater degree resulting in greater inflated balloon that occupies a greater space and has a different degree of conformity to permit the proper seating of the balloon against the landscape of the atrial heart wall.

The operation of the multi-stage inflatable balloon can be by any number of different controllers that permit the user to select a particular setting after the landscape of the atrial wall is investigated as described below. For example, the controller can be in the form of a switch that has multiple positions that correspond to multiple inflation settings and therefore, to select a particular setting, the user simply manipulates the switch and places it in the appropriate desired position. Alternatively, the controller can be in the form of a knob that can be rotated into a plurality of different positions which correspond with multiple inflation settings. In addition, the controller can be part of a digital display unit where the user can use a keypad or the like to enter a particular setting that corresponds with one inflation setting. For example, the settings can be identified by different inflation setting numbers or can be identified by different pressure settings. It will be understood that the user can easily change the selected setting by readjustment of the controller to cause the balloon to be inflated at a different inflation setting (level).

By having a multi-stage inflation balloon, the balloon of the present invention is not part of a “one size fits all” balloon system, but rather, the inflation of the balloon can be customized or tailored to a particular user. This permits a single balloon to be able to be used for all patients and greatly simplifies the process and further, yields to much improved results due to better contact between the balloon and the target area.

As described herein, the compliant balloon can be inflated using any number of different techniques, including by adding a volume of fluid (e.g., a liquid) to the balloon to increase the pressure thereof using conventional techniques, such as the use of a pump or the like. The pump is of a controllable type and communicates with a controller that allows the degree of inflation (e.g., flow of liquid into the balloon) can be precisely controlled. The controller can therefore be configured so that the various inflation settings correspond to different volumes of inflation fluid being delivered into the balloon. For example, there can be five settings numbered 1-5 with setting 1 corresponding to the lowest degree of inflation and setting 5 corresponding to the highest degree of inflation (fully inflated). The settings 2-4 represent intermediate inflation stages.

It will be appreciated that if the inflation setting is not optimal as evidenced by information gathered by the endoscope, the user can adjust the inflation setting of the balloon by adjusting the controller or other user interface resulting in either greater inflation or less inflation of the balloon. Continued observation of the contact between the balloon and target area by the endoscope permits the user to confirm that the selected balloon inflation setting is optimal for the present application and in particular for the patient's heart wall landscape.

It will be appreciated that in the different inflation stages, the balloon will have different inflation characteristics and therefore can be customized for a particular patient. Due to the “dial a size” nature of the present balloon, a single balloon construction can be used to treat all patients. In combination with'the visualization tools of the present invention, including the endoscope, etc., the present invention permits optimal contact to be achieved and maintained between the balloon and the target area (atrial wall).

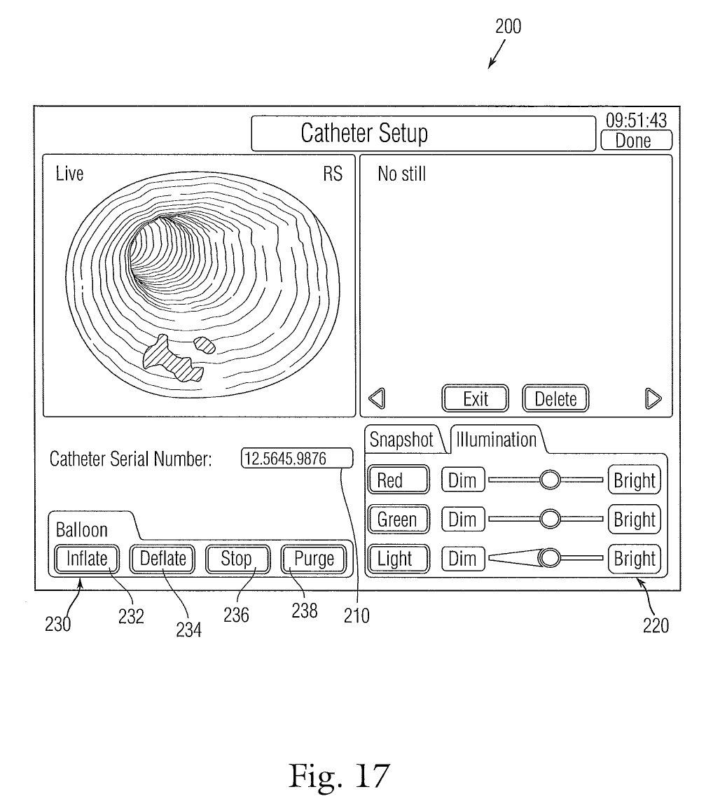

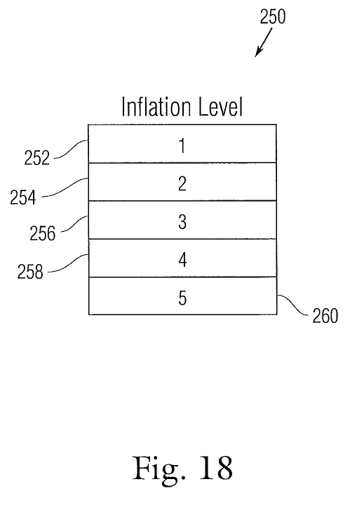

FIGS. 17 and 18 show an exemplary user interface 200 that provides the user with different controls that allow a customized setup of the catheter. In particular, FIG. 17 illustrates a catheter setup screen that includes catheter identifying information, such as a box 210 for the catheter serial number and further as described herein, a live (real-time) view of the surgical site is displayed. The interface 200 includes other controls such as illumination controls, generally indicated at 220. As shown, these controls 220 permit the user to adjust the brightness of the various illuminating members that can be used in accordance with the present invention. For example, the user can adjust the brightness of the aiming light described herein.

The interface 200 also includes a control panel or controls 230 for controlling the operation of the inflation of the balloon (e.g., as by starting a cooling pump and delivering fluid into the balloon). For example, the controls 230 can include various buttons to perform different operations on the balloon. For example, a first button 232 (labeled “Inflate”) is used for inflating the balloon; a second button 234 (labeled “Deflate”) is used for deflating the balloon; a third button 236 (labeled “stop”) is used for stopping the inflation/deflation process; and a fourth button 238 (labeled “Purge”). The buttons 232, 234, 236, 238 can be push buttons that activated by simply pressing the button. In accordance with one embodiment of the present invention, multiple inflation states (levels) can be achieved by simply pressing the Inflate Button 232 successively. In other words, a first pressing of the Inflate Button 232 results in the balloon 26 being inflated to the first level; a second pressing of the Inflate Button 232 results in the balloon 26 being inflated to the second level; a third pressing of the Inflate Button 232 results in the balloon 26 being inflated to the third level, etc. In other words, successive pressing of the Inflate Button 232 results in increased inflation of the balloon.

In yet another embodiment, as shown in FIG. 18, after pressing the Inflate Button 232, the user can be presented with a sub-menu or pull-down menu 250 where different inflation states are selectable. For example and as shown, the pull-down menu can contain a plurality of selectable inflation states, namely, a first inflation level 252, a second inflation level 254, a third inflation level 256, a fourth level 258, and a fifth level 260, etc. Various indicia can be supplied in the buttons that correspond to the different levels. For example, indicia that relates to the pressurization values of the balloon can be displayed. In addition, the level can be displayed in terms of the current level of inflation relative to the maximum inflation. For example, level 1 can be a 20% inflation; level 2 can be a 40% inflation, level 3 can be a 60% inflation, etc.

It will be understood that the aforementioned inflation controls are merely exemplary and other inflation techniques can be used so long as the balloon has multiple inflation levels that are selectable by the user.

The deflation button 234 is depressed when deflation of the balloon 26 is desired as occurs after completing a particular procedure or when repositioning of an inflated balloon 26 is needed. The stop button 236 can be pressed when the inflation process or deflation process needs to be stopped.

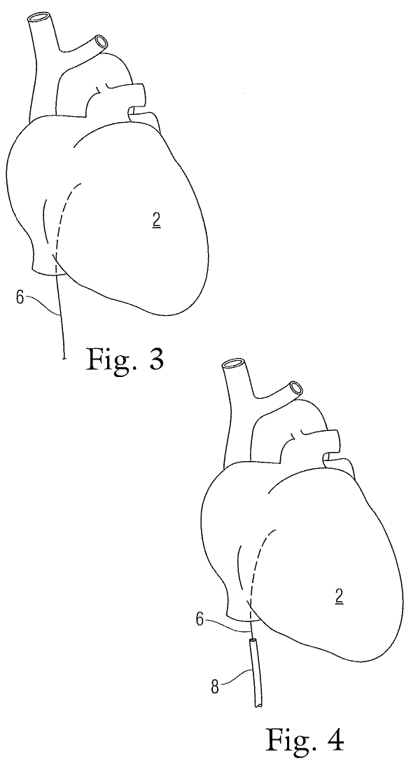

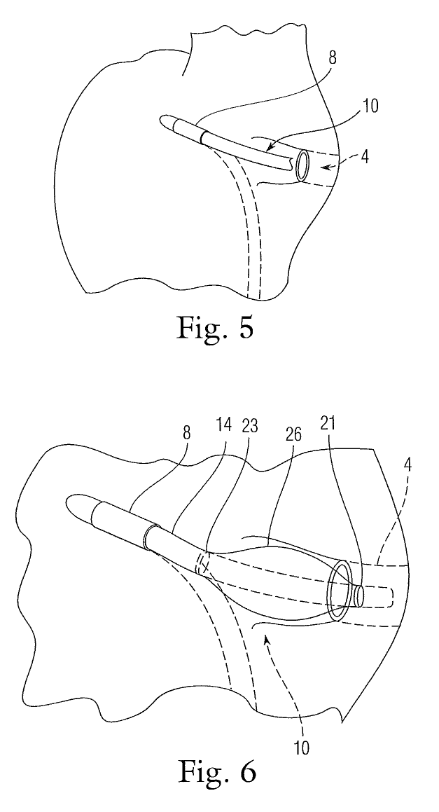

FIG. 3 is a schematic illustration of an initial step in performing ablative surgery with radiant energy according to the invention, in which a guide wire 6 is introduced into a heart 2 and passed into the left atrium. FIG. 4 is a schematic illustration of a method of performing ablative surgery with radiant energy according to the invention. After guide wire 6 is introduced into a heart 2 and passed into the atrium, a deflectable sheath catheter 8 is slid over the guide wire 6. The sheath catheter can be advanced, for example, into the left atrium of the heart. The guide wire can then be withdrawn and replaced with a percutaneous ablation instrument 10 according to the invention as shown schematically in FIG. 5. The catheter 10 can then be advanced to a position proximal to the ostium or mouth of a pulmonary vein, as shown in FIG. 6, where its balloon element can be inflated. For this purpose, the catheter 10 can further include at least one internal fluid passageway (not shown) for inflation of the balloon 26, which is sealed to the body of the catheter 10 by distal seal 21 and proximal seal 22, such that the introduction of an inflation fluid into the balloon 26 can inflate the balloon.

With reference again to FIG. 2B, the balloon 26 is inflated to define a projection pathway for radiant energy ablation of cardiac tissue. The expanded balloon defines a staging through which radiant energy can be projected in accordance with the invention. In one preferred embodiment, the projection balloon is filled with a radiation-transmissive fluid, such as deuterium oxide (so-called “heavy water”) so that radiant energy from an energy emitter can be efficiently passed through the instrument to a target region 52 of cardiac tissue.

In another aspect of the invention, spot lesions are formed by applying radiant energy to target tissue in a range from about 50 Joules/cm² to about 1000 Joules/cm², or preferably from about 75 Joules/cm² to about 750 Joules/cm² In certain embodiments, the power levels applied by the energy emitter can range from about 10 Watts/cm² to about 150 Watts/cm² and the duration of energy delivery can range from about 1 second to about 1 minute, preferably from about 5 seconds to about 45 seconds, or more preferably from about 10 to about 30 seconds. For example, for power levels between 10 and 75 Watts/cm² it can be advantageous to apply the radiant energy for about 30 seconds. Lesser durations, e.g., of 10 to 20 seconds, can be used for power levels of 75 to 150 Watts/cm².

The balloons described herein can be preshaped to form parabolic like or various other shapes (e.g., to assist in seating the instrument at the mouth of a pulmonary vein or otherwise engaging the vein ostium or other anatomically defined regions of the heart). This can be accomplished, for example, by shaping and melting a polymeric film in a preshaped mold to effect the desired form. Compliant balloons according to the present invention can be made, for example, of thin wall polyurethane with a membrane thickness of about 5-50 micrometers, and, in some applications, preferably less than 10 micrometers or less than 5 micrometers in thickness in an inflated state. The balloon is also preferably an elastic or pliable material, e.g., with a durometer ranging from about 35 A to about 55 D, or preferably from about 75 A to about 95 A, as measured according to the Shore durometer scales.

It should be noted that it is not necessary for the balloon 26 to contact the target tissue in order to ensure radiant energy transmission. One purpose of the projection balloon is simply to clear a volume of blood away from the path of the energy emitter. With reference again to FIG. 2B, an ablative fluid can be employed outside of the instrument (e.g., between the balloon 26 and the target region 52) via irrigation ports (not shown) to ensure efficient transmission of the radiant energy when the instrument is deployed. The ablative fluid in this context is any fluid that can serve as a conductor of the radiant energy. This ablative fluid can be a physiologically compatible fluid, such as saline, or any other non-toxic aqueous fluid that is substantially transparent to the radiation. The fluid can also serve an irrigation function by displacing any blood within the path of the radiant energy, which could otherwise interfere with the radiant light energy transmission to the target region 52.

For alternative designs for delivery of ablative and/or irrigation fluids, see commonly-owned, U.S. patent application Ser. No. 09/660,601, filed Sep. 13, 2000 entitled “Balloon Catheter with Irrigation Sheath,” the disclosures of which are hereby incorporated by reference. For example, in one embodiment described in patent application Ser. No. 09/660,601, the projection balloon can be partially surrounded by a sheath that contains pores for releasing fluid near or at the target ablation site. One of ordinary skill in the art will readily appreciate that such pores can vary in shape and/or size. A person having ordinary skill in the art will readily appreciate that the size, quantity, and placement of the fluid ports of various designs can be varied to provide a desired amount of fluid to the treatment site.

In one illustrated embodiment, shown in more detail below, the energy emitter 40 can be a radiant energy emitter and includes at least one optical fiber 42 coupled to a distal light projecting, optical element 43, which cooperate to project a spot of ablative light energy through the instrument to the target site. The optical element can further comprise one or more lens elements and/or refractive elements capable of projecting a spot or arc-shaped beam of radiation. Alternatively, the lesion generator may generate a annulus or partial ring of ablative radiation, as described in more detail in commonly owned U.S. Pat. No. 6,423,055 issued Jul. 22, 2002, herein incorporated by reference.

In one preferred embodiment, the optical element is adapted to project an arc-like pattern of radiation and the energy emitter can be rotated and/or translated to encircle the pulmonary vein. Alternatively, the radiant energy emitter can be an ultrasound or microwave energy source, as described in more detail below that is likewise configured to generate a series of vein-encircling spot lesions.

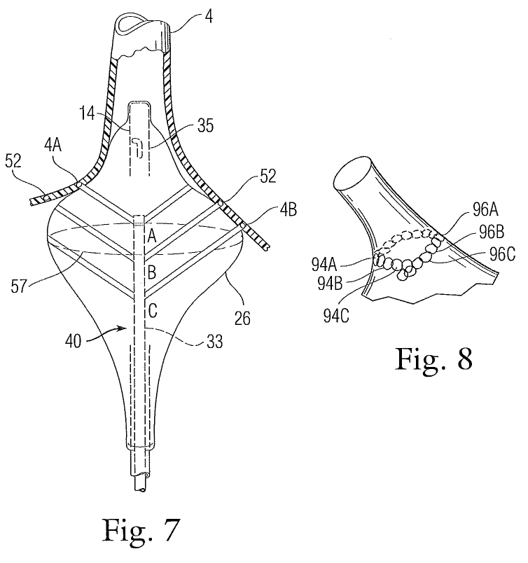

FIGS. 7 and 8, taken together, also illustrate an advantageous feature of the present invention, namely, the ability to select the location of a lesion independent of the instrument design or location. Because the radiant energy emitter does not require contact with a target tissue region and is, in fact, decoupled from the rest of the instrument, the present invention permits the clinician to select a desired target region by simply moving the emitter (e.g., within the lumen of the catheter). As shown in FIG. 7, the radiant energy emitter can be positioned to form a wide circumferential lesion (when the shape of the pulmonary vein ostium warrants such a lesion) by positioning the radiant energy emitter at the rear of the projection balloon—at a distance from the target tissue denoted as “C”. Alternatively, a smaller diameter lesion can be formed by positioning the radiant energy emitter closer to the front of the projection balloon, as shown in positions “A” or “B”. Smaller lesions can be preferable when the geometry of the vein ostium presents a sharper change in diameter, as shown by schematic wall segment 4B. It should be appreciated that it may be desirable to change the intensity of the emitted radiation depending upon the distance it must be projected; thus a more intense radiant energy beam may be desirable in the scheme illustrated in position “C” in comparison with position “A.”.

In addition, the expandable element can include one or more orientation markers 57 that can be visualized endoscopically, to aid in determining the location of tissue contact or targeted energy delivery relative to geometric features of the expandable element. For example, the orientation marker 57 may take the form of an endoscopically visible circumferential line on the portion of the expandable element that generally presents the largest diameter upon inflation. In the endoscopic view this line appears as circle. This circles acts as an aide in determining the optimal amount of energy to deliver to a given portion of tissue when the tissue is viewed endoscopically. When energy is delivered substantially near the circle as seen in the endoscopic view the operator will select a larger amount energy since energy delivery near the circle is know to be energy delivery substantially near the maximum diameter of the expandable element. If energy is delivered substantially outside of or substantially inside of the circle the user will know to deliver a lower amount of energy since such deliveries are substantially near the smaller diameter proximal and distal portions of the expandable element respectively. In a similar manner the expandable element may contain a series of circumferential lines corresponding to generally differing diameters and appearing as generally concentric circles in the endoscopic view each circle being associated with an amount of energy generally corresponding to the associated diameter of the expandable element. These concentric circles may also be radiopaque and visible fluoroscopically as a further aid to understanding the geometric relationship of the balloon to the complex left atrial anatomy.

The energy emitter 40 and catheter body 14 can each include one or more markers (shown schematically as elements 33 and 35 respectively) to aid in determining the location or tracking movements of the elements. Markers 33 and 35, for example, can be radiopaque lines that can visualized fluoroscopically.

In addition, the expandable element or the catheter body can include an orientation marker 35 which can be visualized both fluoroscopically and endoscopically or alternatively the catheter body or expandable element may include an orientation marker that can be visualized fluoroscopically and whose location relative to a characteristic feature of the device which can be seen endoscopically is known. These markers are used to visualize the position and/or rotational orientations of the catheter body relative to the patients anatomy. Each of the markers 35 can be suitably shaped to provide rotational information when viewed fluoroscopically. For example, the markers can be shaped in the form of an “L” to assist in understanding the rotational orientation. Once the rotational orientation of the catheter body is know the endoscopic view may be rotated either by electronically manipulating the video image or by rotating the endoscope fiber relative to the video camera. The goal of rotating the image is to achieve an orientation such the user may understand where on the endoscopic image the superior, posterior, inferior etc. aspect of the pulmonary vein is located. This information is important for two reasons. First two anatomical structures to be avoided during pulmonary vein ablation are the phrenic nerve and the esophagus. Both these structures are located near the posterior portion of the left atrium. Generally lower amounts of energy are preferred to be used when ablating the posterior regions of the veins. Alternatively scrupulous attention to output temperature probes typically placed in the lumen of the esophagus is paid when ablating the posterior aspect of pulmonary veins. Secondly the anterior aspect of some pulmonary veins such as the right superior pulmonary vein are know to generally be thicker than other parts of the veins an consequently a higher energy dose is desired in these regions. Various other marker mechanisms, such as magnetic, capacitive or optical markers, can also be used. The deflectable sheath used to introduce the catheter can be similarly marked to assist in fluoroscopic observation.

If the catheter itself is constructed of a low modulus material, it may be desirable to reinforce it to make more rotationally stable. For example, the catheter can be reinforced by one or more longitudinal rib elements or a braid layer so that it can be more easily “torqued” to overcome endoscopic blind spots or other achieve a desired orientation.

Typically with prior art devices, the target site, e.g., the ostium of a pulmonary vein, can only be located by fluoroscopic inspection during injection of a contrast medium into the vein. Such images are transient. Location of the ablation catheter itself, even with radiopaque markers, is likewise difficult because of the geometry of the heart. Moreover the heart's structure is largely invisible during fluoroscopic inspection.

Endoscopic guidance systems coupled with the use of orientation markers can help overcome these problems. The use of radiopaque markers on the endoscope and/or the catheter allow the user to orient the ablation instrument relative to the pulmonary vein and permit anatomical features seen via the endoscope to be combined with fluoroscopic information. Orientation markers, such as elements 33 and 35 can be used to determine the angular position of the instrument relative to structures such the ostia and also provide a measure of how far a movable element, such as the energy emitter 40, has been advanced within the instrument. (It should be appreciated that numerous other marker schemes can be employed to achieve these objectives, including ring markers on either the energy emitter and/or the catheter body.)

Similarly, the ring marker shown as element 57 on the projection balloon 26 can be replaced by a series of rings. Alternatively, if the endoscope is maintained in a fixed position relative to the balloon, physical markers can be replaced with virtual markers generated electronically as part of the display. Such information is particularly useful in selection one or more of alternative sites for ablation. In addition to the movable energy emitters described herein, the invention can be used in conjunction with two or more fixed ablation elements (e.g., resistive heating bands of different circumferences) to select the most appropriate one (or set) of the ablation elements to be activated for lesion formation.

The endoscopic guidance systems of the present invention can further be used to position any movable point source of ablative energy, e.g., a rotating contact or radiant ablation element in lieu of a slidably positionable source or together therewith, such that the desired path for circumferential can be visualized and followed by the ablation element. Most generally, the endoscopic guidance systems of the invention can be used together with various fluoroscopic or other imaging techniques to locate and position any one of the various instruments necessary for cardiac ablation.

The ability to position the energy emitter, especially when radiant light is employed as the ablation modality, also permits endoscopic aiming of the energy. For example, an aiming light beam can be transmitted via the catheter to the target site such that the physician can visualize where the energy will be delivered. Thus, endoscopic guidance permits the user to see where energy will be projected at various locations of the energy emitter. Thus, if the instrument is designed to project light in an annular ring (or arc or spot) around the ostium of a pulmonary vein, the aiming beam can be projected down the same optical delivery path as would the radiant energy. If the “aiming beam” is projected onto a region of the atrium where a clear transmission pathway is seen (e.g., there is continuous contact (or the desired lesion path is otherwise cleared of blood), then the physician can begun the procedure. If, on the other hand, a clear transmission pathway is not seen at a particular location of the ablation element, then the ablation element can be moved until a clear lesion pathway is found.

Although this “aiming” feature of the invention has been described in connection with radiant light energy sources, it should be clear that “aiming” can be used advantageously with any radiant energy source and, in fact, it can also assist in the placement of fixed or contact-based ablation elements. Most generally, endoscope-guidance can be combined with an aiming beam in any cardiac, ablation system to improve positioning and predetermination of successful lesion formation.

The terms “visual,” “visualize,” “observe” and derivatives thereof are used herein to describe both human and machine uses of reflectance data. Such data can take the form of images visible to a clinician's eye or any machine display of reflected light, e.g., in black & white, color or so-called “false color” or color' enhanced views. Detection and display of reflected energy measurements outside the visible spectrum are also encompassed. In automated systems such visual data need not be displayed but rather can be employed directly by a controller to guide the ablation procedure.

As described hereinbefore, the aiming light has a pulsed operating mode in which the visible light from the aiming light unit is delivered in pulses to cause intermittent illumination of the tissue. This gives the aiming light an appearance of being a blinking light. By delivering the visible aiming light in pulses, the operator is able to directly observe the tissue that is being treated, using an endoscope, between the aiming light pulses. In other words, an endoscope is used to determine the extent of tissue ablation by sensing the change in color of the tissue as it is ablated and at a time when the aiming beam is in an off cycle.

In between the blinking (pulses) of the aiming light, the operator can observe the treated tissue and see how the treatment is progressing. More specifically, an endoscope is used to determine the extent of tissue ablation by sensing the change in color of the tissue as it is ablated and at a time when the aiming beam is in an off cycle.

If the operator is not satisfied with the nature of the ablation being formed, the operator can simply redo or continue the treatment of a specific tissue location. Conversely, if the operator is satisfied with the treatment, the operator can move on to a new tissue location to continue the treatment. In addition, once the entire treatment is performed, the pulsing action of the aiming light permits the operator to view all segments forming the entire ablation to see if a continuous, uninterrupted ablation has been formed (or see if the ablation has the intended, desired shape). It will likewise be understood that the user can turn off the aiming light to permit visual inspection of the tissue. If there are visible gaps or other imperfections with the formed ablation, the operator can move the energy emitter to the proper location for retreatment of these areas until the desired ablation is formed.

As a result, the pulse mode of the aiming light overcomes the disadvantages associated with the use of a continuously illuminated aiming light and results in increased ablation success rates due to a more optimal and more accurate viewing experience by permitting the operator to incrementally see the progress, in real time, of the ablation process.

FIG. 7-8 further illustrates the unique utility of the multi-positionable, radiant energy ablation devices of the present invention in treating the complex cardiac geometries that are often encountered. As shown in the figure, the mouths of pulmonary veins typically do not present simple, funnel-shaped, or regular conical surfaces. Instead, one side of the ostium 4B can present a gentle sloping surface, while another side 4A presents a sharper bend. With prior art, contact-heating, ablation devices, such geometries will result in incomplete lesions if the heating element (typically a resisting heating band on the surface of an expandable element) can not fully engage the tissue of the vein or ostium. Because the position of the heating band of the prior art devices is fixed, when it does not fully contact the target tissue, the result is an incompletely formed, partially circumferential, lesion that typically will be insufficient to block conduction.

FIG. 7 illustrates how the slidably positionable energy emitters of the present invention can be used to avoid this problem. Three potential positions of the emitter 40 are shown in the figure (labeled as “A”, “B” and “C”). As shown, positions A and C may not result in optimal lesions because of gaps between the balloon and the target tissue. Position B, on the other hand, is preferable because circumferential contact has been achieved. Thus, the independent positioning of the energy source relative to the balloon allows the clinician to “dial” an appropriately ring size to meet the encountered geometry. (Although three discrete locations are shown in FIG. 4, it should be clear that emitter can be positioned in many more positions and that the location can be varied in either discrete intervals or continuously, if so desired.)

Moreover, the geometries of the pulmonary veins (or the orientation of the projection balloon relative to the ostia) may be such that no single annular lesion can form a continuous conduction block. Again, the present invention provides a mechanism for addressing this problem by rotation and adjustment of the location of the energy emitter to form a series of spot lesions that overlap and create a circumferential block. As shown in FIGS. 14A and 14B, the devices of the present invention can form a first series of lesions 94A, 94B, 94C, etc. by rotation along a first arc when the energy emitter is in a first location and a second series of lesions 96A, 96B, 96C, etc. by rotation along a second arc when the energy emitter is in a second location. Because each spot lesion has an area (dependent largely by the amount of energy deposited into the tissue) the spot lesions can combine, as shown, to form a continuous encircling or circumscribing lesion that blocks fibrillation-inducing electric conduction. Although illustrated as two series of spot lesions along two curved paths, it should be clear that any number of paths (emitter locations) can be chosen in order to complete vein isolation. Moreover, the size of the spots can be varied, e.g., by depositing more energy in a particular location, in the course of a procedure.

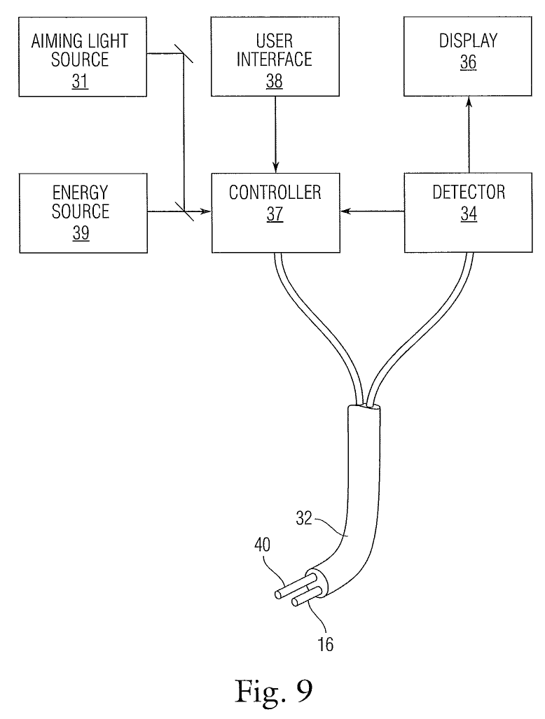

FIG. 9 is a schematic block diagram shown the endoscope/ablator assembly 32 comprising endoscope 76 and ablation element 40 connected to an analyzer system. The analyzer system further includes a detector 34′for detecting reflected light (and preferable for generating a image). The output of the detector 34 can be transmitted to a display 36 for clinician viewing. The display 36 can be a simple eyepiece, a monitor or a heads-up projection onto glasses worn by members of the surgical team. The system can further include an energy source 39, a controller 37 and a user interface 38. In use, the endoscope 76 captures images which can be processed by the detector 34 and/or controller 37 to determine whether a suitable ablation path can be created. An aiming light source 31 can also be used visualize the location where energy will be delivery to the tissue. If a suitable ablation path is seen by the surgeon, the controller 37 can transmit radiant energy from the ablation element 39 to a target tissue site to effect ablation. The controller can further provide simulated displays to the user, superimposing, for example, a predicted lesion pattern on the image acquired by the detector or superimposing dosimetry information based on the lesion location. The controller can further include a memory for storing and displaying data, such as pre-procedure images, lesion predictions and/or actual outcomes. In addition, the displays likewise can show the operator where there are any gaps in the lesion pattern that require the operator to go back and further treat these areas.

The controller can further provide a safety shutoff to the system in the event that a clear transmission pathway between the radiant energy source and the target tissue is lost during energy delivery. In other words, if data and images captured by the endoscope and other equipment show that the transmission pathway is lost due to some event occurring, such as movement of the catheter or loss of contact between the balloon and the target tissue thereby causing blood to flow therebetween, etc.

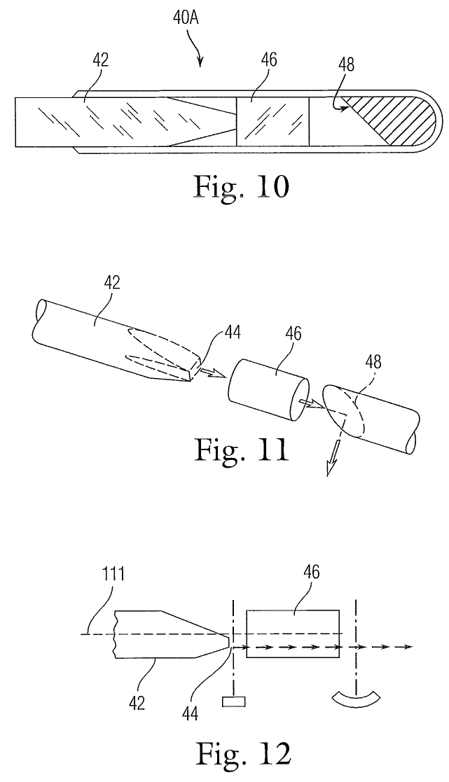

FIG. 10 is a schematic cross-sectional illustration of one embodiment of a radiant energy emitter 40A according to the invention. FIG. 11 is a schematic perspective view of the principal optical components of the emitter. In one preferred embodiment, the radiant energy is electromagnetic radiation, e.g., coherent or laser light, and the energy emitter 40A projects an beam of radiation that forms a spot or arc-shaped exposure pattern upon impingement with a target surface. For example, radiant energy emitter 40A can include an optical fiber 42, the distal end of which is beveled into an energy-emitting face 44 of reduced cross-section. the fiber 42 passes an beam of light to a gradient index (GRIN) lens 46, which serves to collimate the beam, keeping the beam width the substantially the same, over the projected distance. The beam that exits the GRIN lens is reflected by reflector 48 in an angular direction from about 5 degrees to about 110 degrees relative to from the light's path along the longitudinal axis of the catheter. Generally, the angle of reflection from the central axis of the optical fiber 42 can range from about 30 to nearly 90 degrees. In other words, the angle of projection, from the optical axis of the fiber 42 (or lens 46) will be between about 5 to 60 degrees forward of perpendicular.

FIG. 12 is a schematic illustration of a variant on the optical assembly of FIGS. 10 and 11, in which the beveled distal end 44 of the fiber 42 is offset from the centerline 111 of the longitudinal axis, causing light propagating through the GRIN lens 46 to be bent into an arc-shaped exposure pattern. Overlapping patterns of such arc-shaped spots can be used advantageously to form an encircling lesion. The subtended angle of projected annular light, α, can be between about 20 and about 60 degrees, preferably between about 25 and about 35 degrees, most preferably in some applications about 30 degrees.

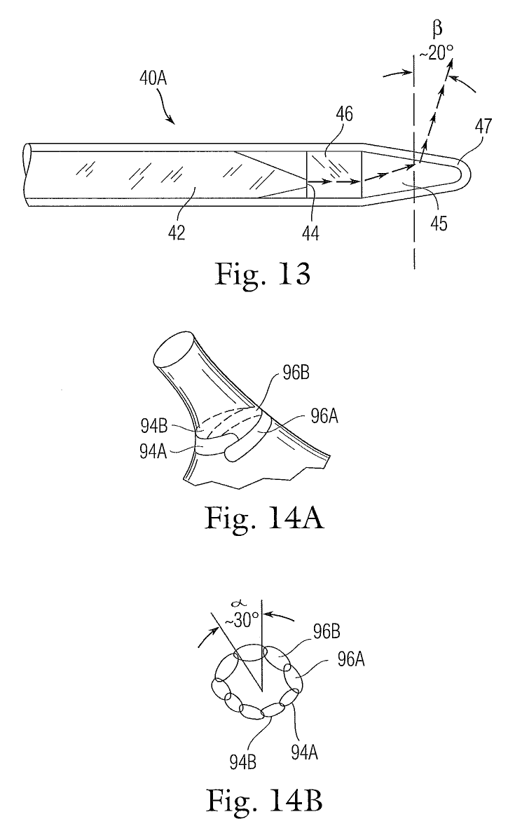

FIG. 13 is a schematic illustration of another embodiment of a radiant energy emitter 40A according to the invention. Again, in this embodiment, the radiant energy is electromagnetic radiation, e.g., coherent or laser light, and the energy emitter 40A projects a beam of radiation that forms a spot or arc-shaped exposure pattern upon impingement with a target surface. For example, radiant energy emitter 40A can include an optical fiber 42 again having a beveled distal tip offset from the central axis. Ablative energy entering the gradient index (GRIN) lens 46 will be formed into an arc-shaped beam of light. The GRIN lens 46 also serves to collimate the beam, keeping the beam width the same, over the projected distance. The beam that exits from the GRIN lens 46 will be refracted first in the air gap 45 of the distal end cap and again as it passed through the wall of the end cap. The projected light beam will expand (in diameter) over distance, but the energy will remain largely confined to a narrow annular band. Generally, the angle of projection, β, from the optical axis of the fiber 42 (or lens 46) will be between about 5 to 60 degrees forward of perpendicular. Again, the subtended angle of the arc-shaped spots can be between about 20 to about 60 degrees, preferably between about 25 and about 35 degrees, most preferably in some applications about 30 degrees.

The diameter of the beam of light will be dependent upon the distance from the point of projection to point of capture by a surface, e.g., a tissue site, e.g., an interstitial cavity or lumen. Typically, when the purpose of the radiant energy projection is to form a transmural cardiac lesion, e.g., around a pulmonary vein, the diameter (or minimum width) of the beam will be between about 6 mm and about 20 mm, preferably greater than 10 mm or greater than 15 mm. In some instances a beam width greater than 20 mm can also be useful. The cross-sectional area of the beam at tissue impingement can range from about 5 mm² to about 500 mm² to form lesions having average surface areas of the same or similar sizes. In some applications is preferable to control the spot size to create lesions having surface areas of less than about 100 mm². When the spot is arc-shaped as shown in FIG. 14A and 14B, the subtended angle of projected annular light is between about 20 and about 60 degrees, preferably between about 25 and about 35 degrees, most preferably in some applications about 30 degrees.

Preferred energy sources for use with the percutaneous ablation instruments of the present invention include laser light in the range between about 200 nanometers and 2.5 micrometers. In particular, wavelengths that correspond to, or are near, water absorption peaks are often preferred. Such wavelengths include those between about 805 nm and about 1070 nm, preferably between about 900 nm and 1100 nm, most preferably, between about 960 nm and 1000 nm. In certain embodiments, wavelengths around 915 nm or or around 960 nm around 980 nm can be preferred during endocardial procedures. Suitable lasers include excimer lasers, gas lasers, solid state lasers and laser diodes. One preferred AlGaAs diode array, manufactured by Spectra Physics, Tucson, Ariz., produces a wavelength of 980 nm.

The optical waveguides, as described in above, can be made from materials known in the art such as quartz, fused silica or polymers such as acrylics. Suitable examples of acrylics include acrylates, polyacrylic acid (PAA) and methacrylates, polymethacrylic acid (PMA). Representative examples of polyacrylic esters include polymethylacrylate (PMA), polyethylacrylate and polypropylacrylate. Representative examples of polymethacrylic esters include polymethylmethacrylate (PMMA), polyethylmethacrylate and polypropylmethacrylate.

Internal shaping of the waveguide can be accomplished by removing a portion of material from a unitary body, e.g., a cylinder or rod. Methods known in the art can be utilized to modify waveguide to have tapered inner walls, e.g., by grinding, milling, ablating, etc. In one approach, a hollow polymeric or glass cylinder, e.g., a tube, is heated so that the proximal end collapses and fuses together, forming an integral proximal portion which tapers to the distal end of the waveguide. In another approach, the conical surface 45 can be formed in a solid quartz cylinder or rod by drilling with a tapered bore.

Waveguide 44 can be optical coupled to optical fiber 42 by various methods known in the art. These methods include for example, gluing, or fusing with a torch or carbon dioxide laser. In one embodiment, waveguide 44, optical fiber 42 and, optionally, a gradient index lens (GRIN) 46 are in communication and are held in position by heat shrinking a polymeric jacket material 49, such as polyethylene terephthalate (PET) about the optical apparatus 40.

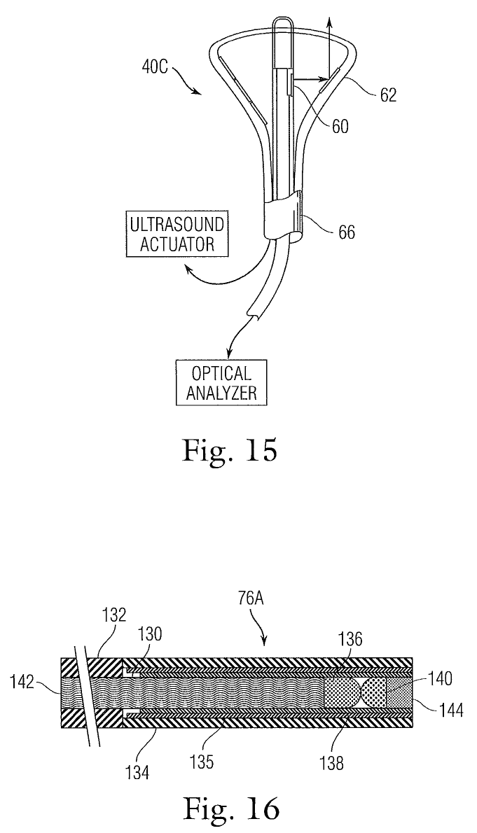

FIG. 15 illustrates an alternative embodiment of a radiant energy emitter 40C in which an ultrasound transducer 60, comprising individual shaped transducer elements (and/or lenses or reflectors) 62 which direct (project) the ultrasound energy into a spot of energy that can likewise form an annular exposure pattern upon impingement with a target surface. The emitter 40C is supported by a sheath 66 or similar elongate body, enclosing electrical leads, and thereby permitting the clinician to advance the emitter through an inner lumen of the instrument to a desired position for ultrasound emission.

Yet another embodiment of a radiant energy emitter can employ microwave energy similarly focused into a spot or arc-shaped exposure beam. The microwave energy emitter can include a coaxial transmission line (or similar electrical signal leads) and a helical coil antenna. Radiation reflectors can cooperate to shield and direct the radiation into a spot. In other embodiments, a radioisotope or other source of ionizing radiation can be used in lieu of the microwave antenna, again with appropriate radiation shielding elements to project a beam of ionizing radiation.

It should be clear that the invention can be practiced with various numbers of illuminating and/or sensing elements, and with or without use of the energy emitter as an element in the contact sensing module. The emitter and the endoscope can each move independently, if desired. Moreover, ultrasound emitters and detectors can also be used in the same manner in lieu of the light reflecting mechanisms to determine contact. In any event, the output signals of the sensors can be electronically processed and incorporated into a display.

The devices of the present invention can further include illumination elements that are capable of diffusing light to a large contact area of tissue by employing a scattering medium at the distal end of the illumination fiber. Examples of this diffusing material can be a matrix of titanium dioxide particles suspended in cured silicone. This diffusing medium allows high intensity light to be uniformly diffused over a large area preferably over an area greater than 40 mm in diameter.

Endoscopes useful in the present invention can include a coherent optical fiber bundle for transmitting the captured image back to a detector and display. The distal end of the endoscope can be coupled to a set of lends which create an image at the distal end of the fiber bundle and provide an enhanced field of view. Such field enhancing elements preferably increase the field of view to greater than 50 degrees, more preferably to about 110 degrees or higher. Typically, commercially available endoscopes have a field of view of about 50 degrees or less in air. However, when immersed in water or similar fluids, the field of view of the endoscope is further reduced due to the refractive index difference between water and air. As explained in more detail below, a greater field of view can be very important to endoscopic guidance.

The endoscopes of the present invention provide the ability to position the percutaneous ablation instruments of the present invention at a treatment site such that proper location of the energy emitter vis-à-vis the target tissue (as well a satisfactory degree of contact between the projection balloon and the tissue) is achieved.

FIG. 16 provides a detailed schematic illustration of an endoscope 76A with enhanced field of view. The endoscope can include a fiber bundle 130 within a protective polyimide tube 132 coupled to distal stainless steel tube 134 in which the field-enhancing optics are disposed. Within distal tube 134, an imaging lens 136 and an objective lens 140 are situated, together with a centering and connecting tubes (e.g., tube 135 and 142) as may be needed to secure the lenses in place. (It should be appreciated that various lens combination or compound lens structures can be substituted for the elements shown in FIG. 15.)

The endoscope 76A is designed to have a wide field of view even while it is immersed in liquid. The liquid in which it is immersed typically can be either physiological saline in the inner lumen of the catheter or deuterium oxide which is one preferred medium for filling applicants' projection balloon. Both of these liquids have essentially the same index of refraction. To achieve the wide field of view a lens system such as shown in FIG. 11A can be used. The lens system consists of two plano-convex lenses 136 and 140 arranged as shown along with an apertured window 144. High index of refraction materials are preferably used for the lenses. Suitable materials include sapphire, cubic zirconia or high index glass materials. Alternatively, air-filled optical structures can be substituted for the solid lenses shown in the figure. All these materials are readily available as small diameter spheres with optical quality surfaces. The spheres can be made into hemispheres and the diameter of the hemispheres are reduced using common lens grinding technology. The aperture can be constructed by metallizing one surface of flat glass plate. The central aperture hole is created by masking the flat glass before the metallization or removing the metallization with a laser.

The ability have a field of view greater that 110 degrees (and, preferably, in some applications, greater than 70 degrees, or 90 degrees) can be important because of the geometry of the heart and the ablation elements. Visualization of the ostium of a pulmonary vein inherently requires a wide field of view. Moreover, the ablation element (including any expandable element) must be short because of the limited space available within the atrial chamber. These two factors combine to require the placement of the endoscope close to the vein and an even wider field of view is desirable, typically greater than 70 degrees, in order to visualize the target region and the instrument's position relative to the target region. Moreover, the wide field of view allows the clinician to see well proximal to the apex of the balloon; thus providing the ability to determine if the instrument is placed too deep in the pulmonary vein.

Thus, formation of ablative lesions for treatment of atrial fibrillations and the like can be accomplished with the instruments and systems described herein by observing balloon location via an endoscope deployed within applicants' catheter devices. For example, the method can include observation of a marker on the balloon to determine orientation of a balloon (or other expandable element) within the heart. In one embodiment, the balloon contains a pair of markers, namely a first marker that is located proximate at or near the proximal end of the balloon and a second marker that is located at or near the distal end of the balloon. The first and second markers can be in the form of circular shaped markers that are visible on the balloon during the procedure as viewed with a visualization tool, such as the endoscope. The first and seconds markers can be used to determine if the balloon is in a desired orientation. More specifically, if in the field of view (e.g., the endoscope), the circular shaped markers appear concentric, the balloon is on center and is properly oriented in that it is straight relative to the reference (tissue). In contrast, when the balloon is off-centered, the circular shaped markers do not appear concentric and instead, the markers may intersect or otherwise not be concentric in nature.

The method can further include projecting an aiming beam during (e.g., while or following) positioning of the energy emitter to identify a candidate site for treatment. Preferably, the aiming beam is substantially coaxial with the treatment energy beam and the spot formed by the aiming beam is coincident (e.g., substantially overlaps) with the site to be ablated. In a preferred embodiment, the aiming beam is introduced via a beam combining mirror arrangement (as shown in FIG. 9) such that the aiming beam is projected via the same optical fiber, lens and reflector elements as the treatment beam. The aiming beam can verify that a candidate site is acceptable for ablation by endoscopic observation of where the aiming beam impinges. Any visible wavelength including white light can be used as the aiming beam, however, in certain applications it can be advantageous to employ two or more discrete wavelengths, e.g. red and green light, to visualizing both tissue contact and blood.

Verification can be based on endoscopic observation of balloon contact with tissue in the vicinity of the aiming beam impingement—or on similar observations of a lack of balloon contact with blood in the vicinity of the aiming beam impingement. The aiming beam can, thereby, be used to determine the topography of the cardiac tissue at a candidate site, assess the quality of contact and/or lack of blood occlusion. Coupled with endoscopic observation of lesions as they are formed, the aiming beam can further be used to predict the path of future therapy and “line-up” the next candidate site to ensure the formation of contiguous (overlapping) spot ablations and, ultimately, a continuous vein-encircling lesion.

Endoscopic observation can also be used to select a dose for forming a spot lesion based on endoscopic observation following expansion of the expandable member and positioning of the energy emitter. For example, a dose can be selected based on an observed size of an impingement spot of an aiming beam or based on an observed balloon location.

The methods of the present invention can further include forming a first spot lesion and then repositioning the energy emitter to a second location. Next, visualization can be used to verify a second candidate site is acceptable for ablation by endoscopic observation of a aiming beam during repositioning of the energy emitter. Such visualization of observation can further be used to determine that a second (or subsequent candidate site) is suitable to forming a subsequent lesion that will be contiguous with the first lesion.

The endoscopes of the present invention can also be used in conjunction with other optical reflectance measurements of light scattered or absorbed by blood, body fluids and tissue. For example, white light projected by an illumination source toward tissue has several components including red and green light. Red light has a wavelength range of about 600 to about 700 nanometers (nm) and green light has a wavelength range of about 500 to about 600 nm. When the projected light encounters blood or body fluids, most if not all green light is absorbed and hence very little green or blue light will be reflected back toward the optical assembly which includes a reflected light collector. As the apparatus is positioned such that blood and body fluids are removed from the treatment field cleared by an inflated balloon member, the reflectance of green and blue light increases as biological tissue tends to reflect more green light. As a consequence, the amount of reflected green or blue light determines whether there is blood between the apparatus and the tissue or not.

Thus, the endoscopic displays of the present invention can incorporate filters (or generate “false-color” images) that emphasize the presence or absence of blood in the field. For example, when the inflated balloon member contacts the heart tissue (or is close enough that the balloon and ablative fluid released by the instrument form a clear transmission pathway), more green light will be reflected back into the optical assembly and the collector. The ratio of two or more different wavelengths can be used to enhance the image. Accordingly, a color-enhanced endoscope can permit visualization of the instrument and/or the target site, as well as a determination of whether blood precludes the formation of a continuous lesion, e.g., circumferential lesion around the ostium of a pulmonary vein.

The system can incorporate and use the above-described reflectance data as part of a safety shut-off feature. For example, the controller can be configured to analyze the amount of reflected light of a particular color in order to determine if the balloon member is in proper contact with the heart tissue. For example, the controller can compare the amount of reflected green or blue light to a threshold value and if the amount of reflected light exceeds a value, this is an indicator that blood is present between the balloon and the tissue and therefore, the balloon is not properly seated. When this event occurs (i.e., the reflected light exceeds a threshold value), the controller can send a control signal to the power source of the energy emitter resulting in shut down of the system. Similarly, the reflectance of red light can be compared to a threshold value as part of an automatic shut off safety feature. An increase in the amount of reflected red light coming back through the energy emitter indicates the presence of blood in the path of the radial thermal energy emitted by the energy emitter and can be used as the automatic shut off safety feature where power is cut off when a threshold value (reflected red light) is reached.

Alternatively, spectrographic measurements can be taken in tandem with endoscopic imaging, Thus, reflected light can be transmitted back through a collector, such as an optical fiber to a spectrophotometer. The spectrophotometer (Ocean Optics Spectrometer, Dunedin, Florida, model S-2000) produces a spectrum for each reflected pulse of reflected light. Commercially available software (LabView Software, Austin, Tex.) can isolate values for specific colors and perform ratio analyses.

Once the operator is satisfied with the positioning of the instrument, radiant energy can then be projected to the target tissue region. If the radiant energy is electromagnetic radiation, e.g., laser radiation, it can be emitted onto the tissue site via a separate optical fiber or, alternatively, through the same optical fiber used for transmitting the white, green or red light. The laser light can be pulsed intermittently in synchronous fashion with the positioning/reflecting light to ensure that the pathway remains clear throughout the procedure.

The apparatus of the present invention thus permits the selection of an ablative lesion, e.g., a circumferential lesion, of desired shape and size. This adjustability can be used advantageously to form a lesion at a desired location, or along a desired path, to effectively block conduction and thereby treat atrial fibrillation.

The current assignee has a U.S. Pat. No. 6,932,809 that covers a shut off feature, which is hereby incorporated by reference in its entirety. The system of the present invention can also be configured so that it includes a number of automatic safety shut-off features that result in the automatic shut-off of the power of the energy emitter. For example, the controller of the present device can generate a control signal for shutting off power to the energy emitter when the balloon placement is incorrect. As shown in FIG. 17, the display that is part of the present system includes a real-time image of the surgical site by means of the endoscope. In this view, the perimeter of the balloon can be seen and when the balloon is placed into contact with the tissue, a ring (circumferential band) is seen where the balloon is in contact with the tissue. The use of the endoscope permits the user to see this circumferential band where there is contact between the balloon and tissue. If the balloon is not properly placed against the tissue, a complete circumferential band will not be observed or an odd angle is observed for the contact between the balloon and the tissue.

Thus, if the balloon is not properly placed against the tissue, different parameters that are monitored and observed can indicate that such an undesirable condition exists and the controller can generate and send a shut-off signal that causes the power to the energy emitter to be cut off.

In yet another embodiment, the radial distance (diameter) of the balloon as observed on the display is analyzed and used as a parameter for selecting the appropriate power level of the energy emitter. For example, if a large radial balloon distance is observed, then the power level of the energy emitter can be increased since the observation of a large radial balloon distance indicates that the balloon is closer to the endoscope and further away from the target tissue. As a result, the power level of the energy emitter can be adjusted accordingly. Conversely, if a smaller radial balloon distance is observed, this is indicative that the balloon is further away from the endoscope and is located closer to the target tissue and therefore, less power is needed.

It will therefore be appreciated that there is relationship between the observed radial distance (diameter) of the balloon and the power level of the energy emitter and the controller can be configured so that if the user tries to increase the power level of the energy emitter above the recommended power level which is selected based on the observed characteristics of the balloon (i.e., the radial distance thereof), then the system can send an alert or warning to the user. In addition, a power limiter feature can be employed where the controller will prevent the user from increasing the power to a level where damage can potentially occur. In other words, the power can be shut off or limited when the user dials in or selects an energy or power level that is too high for the energy power emitter based on the observation of the radial distance of the observed balloon which is indicative of the location of the balloon relative to the target tissue.

An override feature can be provided that allowed power to be restored. For example, a switch or the like or a button can be provided.

It will also be appreciated that the controller and safety shut-off feature can be configured so that an impression of the visual arc shaped lesion and the response of the tissue can be monitored. The use of the endoscope permits the visualization of the target tissue region and in particular, permits the progress of the lesion forming operations (energy emissions) to be monitored and allows for remedial steps to be taken if needed. For example, the response of the tissue can be monitored as the energy emitter acts of the tissue to form lesions, such as the arc shaped lesions. If the lesion is not properly formed, then the user can move the energy emitter back and reapply the radiant energy to the same area.

Although described in connection with cardiac ablation procedures, it should be clear that the instruments of the present invention can be used for a variety of other procedures where treatment with radiant energy is desirable, including laparoscopic, endoluminal, perivisceral, endoscopic, thoracoscopic, intra-articular and hybrid approaches.

The term “radiant energy” as used herein is intended to encompass energy sources that do not rely primarily on conductive or convective heat transfer. Such sources include, but are not limited to, acoustic and electromagnetic radiation sources and, more specifically, include microwave, x-ray, gamma-ray, ultrasonic and radiant light sources. The term “light” as used herein is intended to encompass electromagnetic radiation including, but not limited to, visible light, infrared and ultraviolet radiation.

The term “continuous” in the context of a lesion is intended to mean a lesion that substantially blocks electrical conduction between tissue segments on opposite sides of the lesion. The terms “circumferential” and/or “curvilinear,” including derivatives thereof, are herein intended to mean a path or line which forms an outer border or perimeter that either partially or completely surrounds a region of tissue, or separate one region of tissue from another. Further, a “circumferential” path or element may include one or more of several shapes, and may be for example, circular, annular, oblong, ovular, elliptical, semi annular, or toroidal.

The term “lumen,” including derivatives thereof, in the context of biological structures, is herein intended to mean any cavity or lumen within the body which is defined at least in part by a tissue wall. For example, cardiac chambers, the uterus, the regions of the gastrointestinal tract, the urinary tract, and the arterial or venous vessels are all considered illustrative examples of body spaces within the intended meaning.

The term “catheter” as used herein is intended to encompass any hollow instrument capable of penetrating body tissue or interstitial cavities and providing a conduit for selectively injecting a solution or gas, including without limitation, venous and arterial conduits of various sizes and shapes, bronchoscopes, endoscopes, cystoscopes, culpascopes, colonscopes, trocars, laparoscopes and the like. Catheters of the present invention can be constructed with biocompatible materials known to those skilled in the art such as silicone rubber, polyethylene, Teflon, polyurethanes, nylon, polycarbonate, including blends and copolymers such PEBAX, etc. The term “lumen” including derivatives thereof, in the context of catheters is intended to encompass any passageway within a catheter instrument (and/or track otherwise joined to such instrument that can serve as a passageway) for the passage of other component instruments or fluids or for delivery of therapeutic agents or for sampling or otherwise detecting a condition at a remote region of the instrument. The term “catheter” is also intended to encompass any elongate body capable of serving as a conduit for one or more of the ablation, expandable or sensing elements described herein, e.g., energy emitters, balloons and/or endoscopes. Specifically in the context of coaxial instruments, the term “catheter” can encompass either the outer catheter body or sheath or other instruments that can be introduced through such a sheath. The use of the term “catheter” should not be construed as meaning only a single instrument but rather is used to encompass both singular and plural instruments, including coaxial, nested and other tandem arrangements.

The term “vessel” or “blood vessel” includes, without limitation, veins, arteries, and various chambers or regions of the heart, such as the atria, ventricles, coronary sinus, vena cava and, in particular, the ostia or antrum of the pulmonary veins.

It should be understood that the term “balloon” encompasses deformable hollow shapes which can be inflated into various configurations including spherical, obloid, tear drop, etc., shapes dependent upon the requirements of the body cavity. Such balloon elements can be elastic or simply capable of unfolding or unwrapping into an expanded state. The balloon can further encompass multiple chamber configurations.

The term “transparent” is well recognized in the art and is intended to include those materials which allow transmission of energy through, for example, the primary balloon member. Preferred transparent materials do not significantly impede (e.g., result in losses of over 20 percent of energy transmitted) the energy being transferred from an energy emitter to the tissue or cell site. Suitable transparent materials include fluoropolymers, for example, fluorinated ethylene propylene (FEP), perfluoroalkoxy resin (PFA), polytetrafluoroethylene (PTFE), and ethylene-tetrafluoroethylene (ETFE) or polyester resins including polyethylene teraphathalate (PET).

One skilled in the art will appreciate further features and advantages of the invention based on the above-described embodiments. Accordingly, the invention is not to be limited by what has been particularly shown and described, except as indicated by the appended claims. All publications and references cited herein are expressly incorporated herein by reference in their entirety.

1. A cardiac tissue ablation system, comprising:
an elongate catheter configured to deliver a distal end thereof to a patient's heart;
an expandable member formed at the distal portion of the catheter, the expandable member having an elastic portion configured to conform to the shape of a target tissue region upon expansion;
an energy emitter movably disposed within a lumen of the catheter, the energy emitter configured to deliver radiant ablative energy to the target tissue region; and
a controller that includes a safety shut-off feature for automatically shutting off power to the energy emitter when a prescribed event occurs resulting in power to the energy emitter being stopped.

2. The system of claim 1, wherein the prescribed event is the detection that a clear transmissive pathway between the energy emitter and the target tissue has been lost.

3. The system of claim 1, wherein the prescribed event is when an observed amount of reflected light exceeds a threshold value.

4. The system of claim 3, wherein the reflected light comprises red light.

5. The system of claim 3, wherein the prescribed event is when a clear transmission pathway between the radiant energy emitter and the target tissue region is lost during energy delivery.

6. The system of claim 1, wherein the prescribed event is when there is a determination that the expandable member is not in a proper position at the target tissue region.

7. The system of claim 6, further including an endoscope configured to allow direct visualization of the tissue treatment area and to allow the placement of the expandable member to be determined.

8. The system of claim 1, further including an aiming light that emits visible light that is coincident with the radiant ablative energy, the aiming light having a pulsed operating mode where pulses of visible light are delivered to the target tissue region during delivery of the radiant ablative energy.

9. The system of claim 1, wherein the energy emitter is slidably and rotatably disposed within an inner lumen of the catheter thereby allowing the energy emitter to effectively ablate any of a plurality of regions within the target tissue area.

10. The system of claim 1, wherein the expandable member is a blunt-nosed balloon configured to impede passage of the balloon into a pulmonary vein when the balloon is deployed in proximity to the vein.

11. The system of claim 1, wherein the expandable member is a balloon with a tapered distal end that impedes passage of the balloon into a pulmonary vein when the balloon is deployed in proximity to the vein.

12. A method for treating a cardiac condition with visual inspection of a tissue treatment area comprises the steps of:
delivering an expanding member formed on a distal end of a catheter to a position adjacent a tissue treatment area within a patient's heart, the expandable member having an elastic portion configured to conform to the tissue treatment area;
expanding the expandable member by selecting an initial inflation setting of the balloon thereby allowing the elastic portion of the expandable member to conform to the tissue treatment area;
visualizing the tissue treatment area using an endoscope to determine if the elastic portion of the expandable member is in sufficient contact with the tissue treatment area;
positioning an energy emitter at a first location within an inner lumen of the catheter;
classifying the distance between the energy emitter and the tissue treatment area based on a visualization of the size of the expandable member as seen through the endoscope;
selecting a power level of ablative energy to be delivered based on the classification of the distance between the energy emitted and the tissue treatment area; and
delivering ablative energy, having the selected power level, from the energy emitter to the tissue treatment area.

13. The method of claim 12, wherein the step of classifying the distance comprises observing a diameter of the expandable member and selecting the power level based on the observed diameter.

14. The method of claim 13, wherein the greater the observed diameter of the expandable member, the greater the required power level of the ablative energy.

15. The method of claim 12, wherein the step of expanding the expandable member comprises the step of pressing an inflation button that is presented on the display, wherein repeated pressing of the inflation button results in increased inflation of the balloon.

16. The method of claim 12, further including the step of visualizing the tissue treatment area using an endoscope to select a candidate site of the tissue treatment area for positioning the at least partially inflated balloon into contact with the tissue treatment area.

17. The method of claim 12, further including the step of: projecting a visible aiming beam during positioning of the energy emitter, the ablative energy being coincident with the aiming beam, wherein the aiming beam comprises pulsed visible light to allow intermittent visual inspection of the first spot lesion at times when the aiming beam is not projected onto the tissue treatment area, thereby allowing visual inspection of the ablative characteristics and sufficiency of the first spot lesion.

18. The method of claim 12, further comprising repeating the positioning step and the delivering step until a plurality of spot lesions are provided which form a substantially circumferential lesion.

19. The method of claim 18, wherein the circumferential lesion encircles an ostium of a pulmonary vein.
